(19) United States
(12) Patent Application Publication
(10) Pub. No.: US 2020/0041474 A1
(43) Pub. Date: Feb. 6, 2020

(54) Explosive Detection In Cargo Containers And Packages

(71) Applicant: Greg D. Shaffer, Verona, NJ (US)

(72) Inventor: Greg D. Shaffer, Verona, NJ (US)

(21) Appl. No.: 16/055,600

(22) Filed: Aug. 6, 2018

(51) Int. Cl. G01N 33/00

(52) U.S. Cl. CPC G01N 33/0057

(57) ABSTRACT

A system for preventing explosions in a transported object which includes containers and packages with an imbedded port or window, a semipermeable cloth or filter layer that promotes air flow through natural physical and gaseous processes and access to internally generated trace particles and vapor plumes from contained explosive material, the integrity of which is protected by a mesh, grill, or woven matrix, and which may be impregnated with colorific luminescent chemicals that fluoresce, darken, or illuminate in the presence of explosive-containing material.

### FIELD OF THE INVENTION

The Explosive Detection System relates to screening air cargo and shipped or transported containers for explosives and other hazardous materials.

### Background—Prior Art

On Sep. 11, 2001, terrorists boarded four commercial passenger planes and flew them into the World Trade Center Towers and the Pentagon. Despite all the subsequent safety measures, a major loop hole continues to exist. All the attention focused on the screening of passengers and their luggage. Billions-of-dollars and countless hours have been allocated to safeguard against the destruction of commercial passenger planes and their use as weapons of terror. The absence of a comprehensive screening system for explosives and other hazardous materials potentially hidden in air cargo, as well as port containers, luggage, parcels, mail, remains a major vulnerability to our society and in particular, the current air travel security system and transportation of freight.

Passenger planes transport as much as 22% of all airfreight. Cargo comprises 50 percent of a passenger plane's load. Approximately 500,000 passengers are on these daily flights. Any one of these loads in a single package can cover the terrorists' weapon of choice—explosives.

This vulnerability continues to exist for one reason—Air Cargo on passenger planes is not routinely and consistently screened for explosives.

The air cargo sector is growing 9 percent each year. On the other hand, the basic procedure is to call paper work requirements imposed on shippers “screening.” This is known as the “known shipper rules,” since titled “Known Shipper Plus” program. Bills of lading and detailed manifests are required.

The safety of the passengers on these passenger/cargo flights rests with an “honor system.” All information and the existence and identity of the shippers and the freight forwarders are requested. Compliance is voluntary. According to the General Accountability Office, known shippers often fail to provide the basic information. By some estimates, the number of “known shippers” represents only half of the actual number.

Even with 100% compliance, there is no evidence that simply because a shipper is “known” in identify, it can be trusted not to employ potential terrorists or to be created and run for the purpose of terrorism. At 100% compliance, the “system” of screening is still fatally flawed and inadequate.

The shippers and the airlines consistently oppose any changes. They paint a picture of such sheer volume that the imposition of a comprehensive screening system would virtually shut down the system and destroy a multi-billion dollar economy; with a concurrent devastating impact on the nation's total economy. Nevertheless, each day we face the prospect of an equally devastating impact of similar economic proportions and horrific further loss-of-life from a mid-air terror-caused cargo hold explosion

Beyond the human toll, the costs of screening air cargo pale in comparison to the costs of not screening this cargo. Presently, airline insurance costs are billions-of-dollars for a year, six times higher then pre September 11th levels. A single cargo terror event will drive these costs even higher. Experts say that a single air cargo event exploding in our skies would result in halting of air traffic, the deterring of air travelers, and losses in local businesses and tourism costing the economy $500 billion. The American public who flies on these passenger planes transporting cargo is unaware of the risk that they take each and every time they fly. Such an event and the subsequent knowledge that the screening they endured was useless, because cargo transported in the hold was not screened, would certainly result in a devastating loss of trust and a significantly escalated fear of flying.

Industry and Government leaders have had warnings of potential air cargo catastrophes for many years. Dec. 21, 1988, Pan-Am Flight 103 exploded and pieces of the plane fell onto the Scottish town of Lockerbie, killing 259 people on board and 11 people on the ground. This tragic attack should have alerted government and the industry to the potential for attack with explosives hidden in cargo. Little has been done in response.

The subsequent investigation under the chairmanship of Senator Al Gore revealed the dangers of air cargo. The investigation determined that the Flight-103 explosion was caused by plastic explosives hidden in a radio placed in luggage in the cargo hold.

The General Accounting Office (GAO), Congress's investigative arm, reported that passenger airline cargo is extremely vulnerable to terror:
“Commercial airlines remain vulnerable to terrorist attack, almost 50% of each airplane's belly is filled with cargo that receives little or no screening for explosives”

The aviation security law passed after September 11^{th} requires an air cargo screening system. To date, a critical risk-based assessment has not been completed. The threat has been identified, but the attempts to identify high-risk cargo for inspection has not been finalized. A critical target assessment is not completed. And, the method of screening the cargo remains indeterminate.

“This is the soft underbelly that remains in the transportation system” said Senator Diane Feinstein. “It would be so easy to slip something into air cargo and have that cargo explode on an airliner.” she added.

Senator Edward Markey (D-MA) has pushed for a much stronger bill without success. He wants one hundred percent of the cargo placed on to passenger air planes to be screened for explosives. He faces fierce opposition from the cargo industry trade associations. They claim Markey's proposals are not workable since “it would take away the speed and this in turn would take away the motivation for customers to use them.”

In today's post 9/11 world, airline passengers, pilots and flight attendants are subjected to intense, even intrusive, screenings. These security precautions are based on a belief that no one is above scrutiny and no better substitute exists for individualized hands-on inspections.

The same standards do not apply to the cargo carried in the bellies of passenger planes. Though the shipments have long been recognized as tempting targets for terrorists, cargo is transported based on an honor system. Under current Federal security procedures, airlines trust that known companies will ship safe packages rather than inspect the interior of cargo.

The potential dangers posed by the known shipper program have been documented by several government studies. It warned that tougher baggage screening rules would tempt terrorists to target air cargo.

Transportation Security Administration inspectors have found “numerous security violations” during routine checks line cargo handling facilities and other companies involved in the movement of freight, according to a report by the General Accounting Office (GAO), the investigative arm of Congress.

Background checks of those who work for shippers and other firms that handle cargo are inadequate, according to government reports and critics of the current system.

Opportunities for tampering exist because of the way cargo is transported to airports and is handled there. In addition, the large amounts of theft that occur are indicative of the security problems the GAO found. The fact that annual losses to theft total billions demonstrates how lax the security is surrounding air freight.

In NEWSWEEK Jonathan Alter addressed the air cargo question: “ . . . . As they empty their pockets and throw away their bottled water, passengers assume the rest of what's on board has been screened too. Not so.” Passenger Airlines ship nearly 2 billion tons of commercial cargo annually, and they don't have machines that can handle the varied shape and content of so many packages. Less than 7 percent is physically screened. It must be noted that is barely any increase since September 11. Instead they continue to rely on the “Known Shipper” program.

Alter reports that “overseeing thousands of companies is impossible and the data base of those packages with ‘elevated risk’ is problematic.” He quoted Markey “On this cargo thing they are praying; it's a faith-based homeland security.” The other shocking thing he reported is that Congress rejected the September 11th Commission recommendation that each passenger airliner contain at least one hardened container for suspect cargo.

A report prepared by the Federal Aviation Administration's office of aviation security research and development, warned of “catastrophic” flaws in the air cargo security system. The Washington Post quoted an internal TSA report: “Cargo is likely to become and may already be a primary threat vector in the short term.”

Still, another TSA document placed the risk of a terrorist attack via cargo as high as 35 to 65%. According to an internal assessment, TSA needs to improve [cargo] security and reduce risk as soon as possible,” the Post reported. As much as 40% of air cargo is shipped on passenger planes. Only a negligible percentage of cargo is currently screened. And, there is broad agreement among experts and officials that, nothing in the current system prevents determined terrorists gaining access to passenger airbound cargo and planting an explosive device.

While pressing and relatively unaddressed, vulnerabilities to air cargo from explosives and other hazardous materials are not the only vulnerabilities to explosives and hazardous materials that airlines face. Luggage is passed through x-rays scanners, but it is up to security, those often paid as little as possible, to interpret the shapes in the luggage as dangerous or not. As TSA studies and journalistic reports reveal, there have been repeated and systematic failures of airport security to prevent guns, knives and mock explosives in luggage from passing through security.

Some luggage, clothing, and electronics are pulled aside and swabbed for explosive detection analysis. The swabs are then used in ion mobility spectrum analysis machines that vaporize any potential explosive particles and electronically compare them to the molecular weights of known and cataloged explosives. Even were there no false positives, the procedure adds significant and potentially prohibitive delays. Furthermore, it is not systematic. Some items are analyzed, most are not.

### Currently Used Technology

The Known Shipper Plus program does not reliably screen air cargo. It is voluntary and poorly adhered to, according to the General Accountability Office. The federal government cannot say how many shippers there are and in fact, may be wrong by as much as 50%. The effort to screen air cargo by determining which “elevated risk” cargo lacks a criteria, let alone methodology.

Technology for preventing an explosion is little closer now than it was in 2001. Blast-proof air cargo containers add cost-prohibitive weight to the planes, especially with escalating fuel costs. Furthermore, blast-proof air cargo containers have limited effectiveness containing an explosive air cargo explosion.

Today's current explosive detection technology employs bulky, heavy mechanical/electronic devices including x-rays and ion mobility spectrum analysis which is strongly disfavored by the Air Cargo Industries and the Airlines with its trade associations. These devices require each package to travel through a single portal which requires a significant and a prohibitive amount of time. Certain technologies require human interpretation, currently used to screen luggage, reveals the certainty of human error. Freight forwarders utilize x-ray technology that interprets shapes and material content by computer algorithms that are frequently wrong and subject to false alerts which shut down the system and require emergency inspection teams. The other technologies are based upon the presence of vapors or trace particles of explosive or other hazardous material on the outside of the container/package. These trace and vapor detection methods also have false alarms and do not access the interior of the container where the explosive and hazardous material reside.

### The Sirius Solutions Explosive Detection System: Advantages and Objectives

It is an objective of The Sirius Solutions Explosive Detection System to determine whether or not explosives or hazardous materials are concealed within a piece of air cargo, luggage, container, or package. It is another objective of The Sirius Solutions Explosive Detection System to form a port in one or more sides of a piece of air cargo, luggage, or container. It is another objective of The Sirius Solutions Explosive Detection System to form a port in one or more sides of a piece of air cargo, luggage, or container, that permits access to the contents inside. It is another objective of The Sirius Solutions Explosive Detection System to utilize a semipermeable filter layer to capture trace particulates, as air flow is promoted through the layer by natural physical and gaseous processes, to determine whether explosives or other hazardous materials are contained within a piece of air cargo, luggage, or container. It is another objective of The Sirius Solutions Explosive Detection System to determine whether explosives or other hazardous materials are contained within a piece of air cargo, luggage, or container, with a chemical infused in the filter layer, on or part of the container, that reacts to the presence of vapor plumes or trace particulates from explosive material contents. It is another objective of The Sirius Solutions Explosive Detection System to incorporate a chemical or nanotechnological infusion in the collection layer that detects trace particulates and vapor plumes from explosive or hazardous material contents, creating a visible colorimetric or ultraviolet alert for the outside observer. It is a further objective of The Sirius Solutions Explosive Detection System to use these collection and detection semipermeable ports embedded in one or more sides of the piece of air cargo, luggage, or container, to accurately and consistently detect the presence of explosive materials.

A review of all of the relevant art reveals that the only solution to this problem is what is known in the art as an “on the box detection system.” Access into the package or container is required to detect a well-packaged explosive or other hazardous material consistently. The problem is solved once a system which permits access to the contents of the container is developed in conjunction with appropriate detection sensors.

In contrast to “The Sirius Solutions Explosive Detection System” today's technology is ineffective, costly, and prohibitively unworkable. “The Sirius Solutions Explosive Detection System” utilizes a semipermeable portal device to access the explosives and other hazardous materials within any container or confined space and a chemical and a passive detection system for determining whether the package contains explosives or not. Because it is cheap, simple, and passive in its nature, the system also eliminates such obstacles as human error, false positives, and the failure to appropriate government funding which plague the screening of such items now. “The Sirius Solutions Explosive Detection System” is available and presents a real solution to a potentially catastrophic vulnerability.

### SUMMARY

A system utilizing a port or window imbedded in a box, parcel, or container, a semipermeable filter or clothlike material through which air can pass and which can be saturated with nanowire or an explosive detecting reactive chemical, and a rigid protection of this filter or clothlike window or port cover such as mesh, a grill, venting, or a woven matrix that permits external detection of vapors or capture and collection of trace particulates for visual, spectrographic, aromatic, or molecular weight detection of explosive contents without the need for mechanical, thermal, or radiological excitation.

### BRIEF DESCRIPTION OF THE DRAWINGS

The invention may be understood by reference to the following description taken in conjunction with the accompanying drawings, in which, like reference numerals identify like elements, and in which:

FIG. 1A is a cross-sectional view of a depiction of a model container which may be a box, parcel, or air cargo container with a standard port. The port is protected by a mesh, grill, or a woven matrix. Beneath this grill is a semipermeable layer of cloth, or a filter, of the type through which explosive or hazardous material vapor traces pass or trace particulates adhere.

FIG. 1B is a top view is of a grid which may be the detection layer of the window of the present invention;

FIG. 1C is a top view of the detection layer which may include a multitude of zones potentially having different chemical detectors to provide a comprehensive detection device.

FIG. 2A illustrates a perspective view of the container having a substantially rectangular window;

FIG. 2B illustrates a perspective view of the container having a substantially oval window.

FIG. 3 illustrates the manner in which air cargo is transported in boxes gathered and stacked on a pallet and in a Uniform Loading Device (“ULD”);

FIG. 4 illustrates a Uniform Loading Device, ULD, a form-fitting cargo container used to transport cargo, in which filtered detection ports that may be chemically infused can be embedded.

### DETAILED DESCRIPTION

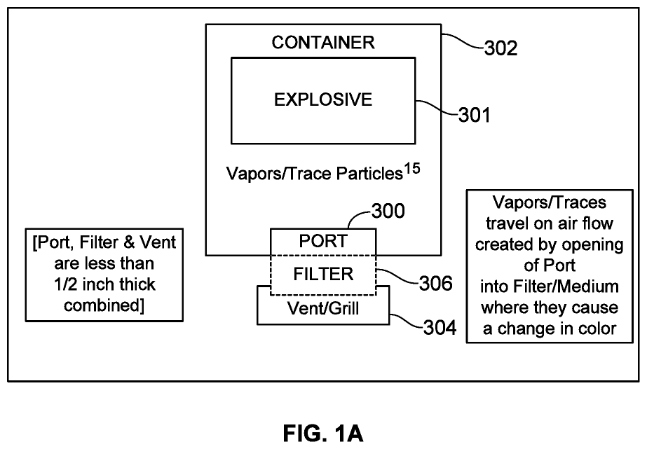

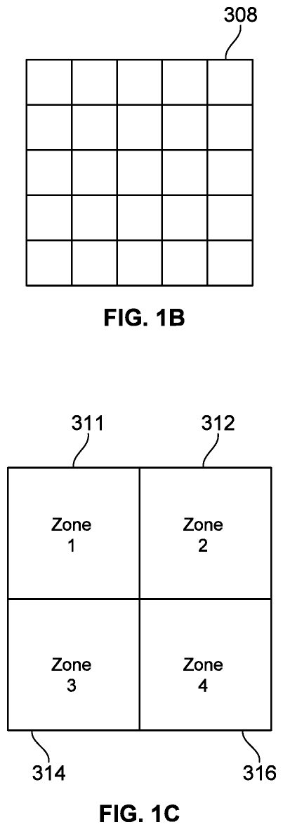

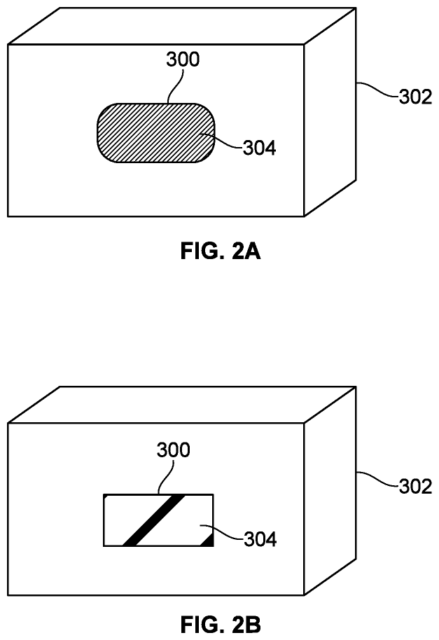

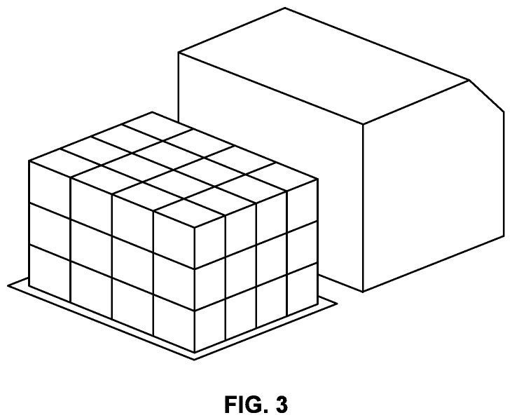

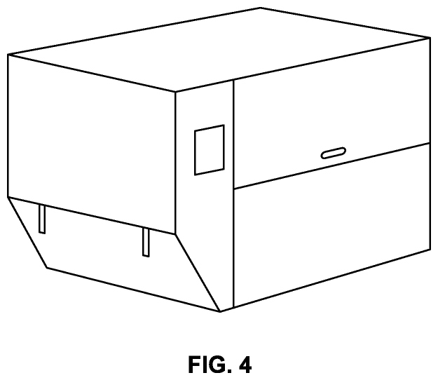

The Sirius Solutions Explosive Detection System as shown in FIGS. 1A, 1B, 1C, 2A and 2B is comprised of any and all packages/containers 302 (which shall include a semi-permeable filter/collection test strip/layer/detection layer 306). The filter collection portion 306 of the cartridge is designed and manufactured with material designed to take advantage of the “stickiness” of explosives and hazardous materials' vapor or trace particulates. The filter collection portion 306 in one or more embodiments, holds or is saturated with one or more chemicals and/or “photoluminescent” substances which reacts with the explosive or hazardous materials vapor or trace residue to create an outline or change of color under normal, black, or ultraviolet light, depending upon the detecting substances utilized in a particular application. In one or more embodiments, the semi-permeable nature of the collection/filter layer 306, which opens the portal to inspection and detection or remains viewable without opening, also allows for detection of vapor or trace particulates of explosives or hazardous materials within the container 302 or confined space by electronic or canine detecting methods outside and separate to the container/confined space.

This trapping/collecting aspect is intended to be compatible and potentially enhanced by the optional use of fans or other airflow enhancing external mechanisms. These methods of optionally increasing airflow are designed to accelerate the air emanating from the containers for increased concentration of trace particulates and vapor identifiers of explosives or hazardous materials within the containers, or mail. Increased internal airflow additionally reveals the opening of the container and the presence of internal airflow by wrinkling or creating a flowing in the drape of the collection/filter layer.

The collection/filter layer 306 may be printed with a counterfeit-proof flat imprint or logo to prevent the replacing of this material with a non-collecting or non-permeable material. It can also be flexible and slack which allows wrinkling of the material when air, such as from an optional blower or ceiling fan, flows against it to verify air flow from the port is not blocked.

The Sirius Explosives Detection System includes TSA mandated or industry practice requiring a container, or package, collectively shown as container 302 with one or more ports/openings in any and all containers. Within the port/window 300 is a filter/trap or semi-permeable material capable of capturing and collecting residue or trace particles and promoting the flow of vapors from hazardous material including explosives. This part of The Sirius Explosives Detection System is well known in the Art and can include PVC netting or Meachem Manufacturing filters depending on the container and usage.

One or more sensor devices, methods, and/or chemical detectors can be used in combination or singly to determine the presence of particular or a full spectrum of hazardous materials in the container/confined area. The preferred embodiment is adaptable to work with a wide variety of explosive detection systems including embedded chemical detectors, external canines, and external electronic/mechanical detectors.

One of these detecting methods features a silicon polymer “nanowire” shown as nanowire grid 308, which will visibly and chromatically change in the presence of explosive traces. In the Sirus Explosives Detection System, the nanowire would be present in the collection material/filter layer contained in the port/opening. In such an application, the portal's material/layer 306, in which the nanowire is embedded/saturated, would turn a darker color, under ultraviolet light or an optical scanner, where and when the explosive detection particulates come in contact with the material. The nanowire's photoluminescent polymers are “quenched” by the presence of such substances as nitroaromatics. See “Polymer Sensors for Nitroaromatics in Explosive Detection,” by Sarah J. Toal and Willaim C. Trogler, 27 Apr. 2006; Journal of Material Chemicals, 206 Vol. 16, p. 2871-2883, by William Trogler, UCSD—incorporated by reference as though set forth completely in this application. With photoluminescent chemicals in The Sirius Explosives Detection System's absorbing/collecting material layer, the presence of explosives, such as TNT, DNT, RDX, HMX, PETN, Otetryl, C-4, PE-4, and Composition B, if inside the container 302, would show through the nanowire-containing material layer 308 of the Sirius Explosive Detection Portal.

RedX has also licensed a separate, handheld “Explosives Particulate Analysis Kit” (an “XPak”) which can be used jointly or independently with chemical detecting substances in the portal's material layer. The kit would be used with trace explosive particles that are captured by the material in the portal, which opens the container or confined space to allow and provoke such particulates to escape.

### Alternative Embodiments

The present invention is a detection system to operate in environments to detect and identify suspect packages having explosive capabilities which may be found in freight/cargo containers and other types of conveyances will:
Be easily operated in airports, seaports, border crossings, government buildings and other venues to rapidly and reliably detect trace particles in containers from hazardous materials;
Provide real-time particulate collection efficiency on any type of package, ULD or container;
Provide continuous monitoring of containers from pre to post shipment phases throughout the supply chain;
Achieve volume monitoring and rapid detection of trace amounts of toxins
Increase duty cycle time in air, sea and rail cargo holds without added manpower;
Avoid the use of large, costly detection machinery and the personnel to operate the costly detection machinery, and
Cost of the detection system will not require government subsidies and will not be borne by the taxpayer.

The present invention will:
allow entry into new markets or a series of markets with a next-generation application detection systems that compliments an entire product line;
increase market share by virtue of new product development

The present invention (SEDS) will quickly detect trace particles/vapors from dangerous substances including chemical, and explosive elements (CBRNE) that pose threats to the public, to business and to industry.

Resulting from the non-mechanical (no moving parts), no-maintenance simplicity, the SEDS device:
Will be easy-to-manufacture;
Provides a low cost, high margin ratio to manufacture and distribute;
Easily incorporates into any package, ULD or container line;
Aids in the reduction of airport, seaport, and other air cargo energy demands that are required to run bulky electronic detection equipment (including those installed at border crossings and foreign seaports);
Is adaptable to multiple embodiments for highly specific detection purposes;
Operates for field-deployable rapid detection purposes;
Offers a cost-effective, non-disruptive system;
Provides an easy-to-interpret product (removes human error)
Solves multifaceted and complex tasks without the aid of additional large equipment.

The SED System:
Provides a device to access dangerous trace substances within any package/container;
Utilizes a passive detection method to rapidly determine whether or not the package/container conceals explosives or other material
Is inexpensive and simple to employ—to raise the level of cargo security
Eliminates obstacles such as human error and mechanical false negatives, and
Bypasses the legislative appropriation process.

The present invention concerning the Sirius Solutions SED portal device provides a non-intrusive, particle imaging inspection technique to automatically detect concealed dangerous warfare agents and other classes of toxic substances that include:
Commercial explosives such as dynamite, Semtex H, TNT, DNT, RDX, HMX, PETN, Otetryl, C4, PE4 and Composition B
Chemicals warfare agents such as chlorine, VX, Sarin and other nerve, blister, blood and toxic industrial chemical threats
Volatile organic chemicals (VOCs) including gasoline, pesticides, and fertilizer
Liquid explosives
Homemade explosive (HME) such as black powder
Chlorates and perchlorates mixed with other substances
Flammable liquid

The present invention provides explosion protection including the following features:
Reliable, instantaneous detection of explosives, bio-chemicals in cargo and baggage;
Restores confidence in flying, eliminates fear of catastrophic terrorist event;
Increased efficiency and regularity of cargo and passenger plane industries;
Eliminates false positives, false alarms, flight cancellations, and airport shutdowns;
Does not stall, slow down or shut down the supply chain;
The present invention is not constrained by limited and time-consuming portal screen machines.

The present invention is cost-effective:
Inexpensive packaging solution v. expensive electronic screening machines;
Private sector solution is provided by the present invention which eliminates competition for government appropriations;
Saves cost of securing supply chain by eliminating inspectors and unnecessary safeguards;
Reduces costly insurance premiums.

### The Portal

The Sirius Solutions Explosive Detection System (SEDS™) port or vent to define a window 300, provides direct and immediate access to the contents of the package/container 301.
This window 300 is positioned on a surface of the container 302 and is covered by a mesh, woven matrix, or a flat vent grate 304 to prevent damage to the window 300 and to maintain the integrity of the window.
Positioned (inside) this window lies a semi-permeable detection layer 306 that “captures” trace particles and liberates vapor plumes from any dangerous substance, transported by air flow promoted by natural physical and gaseous properties.

The portal/window 300 in operation;
Air travels through (both in and out of the container) the port/window 300.
Subtly shifting atmospheric pressure, inertia phenomenon, stabilizing gaseous densities, and air traveling inside and outside the box/container 302, create a small but discernible draft.
When the package/container 302 shifts in position, the air and other particles inside the box/container 302 remain in motion after the shift in position until they collide with the interior of the package/container 302 except at the port/window 300.
There, at the port/window 300, the dangerous particles continue to drift carrying any dangerous substance or explosive's particles and/or vapors that are captured or made accessible by the detection layer 306.

The SEDS Portal System overcomes the following limitations.
The cargo of packages/containers is not screened one at a time.
The packages/containers need not be isolated for inspection.
There is no delay, only rapid detection of dangerous substances.

The features of the detection layer follow:

Nanowire
One embodiment of the SEDS™ Portal System™ utilizes a silicon polymer “nanowire” for the detection layer 306 which may include a nanowire grid 308 which may be formed 2,000 times smaller than the diameter of human hair. This “nanowire” detects trace amounts of such explosive material as TNT and picric acid, a common ingredient used by terrorists to create bombs. The polymer is extremely sensitive to explosives residue and is stable in air and water.

Explosives or Hazardous Materials Detecting Chemicals
There are a number of explosive detection chemical products that may be used for the detection layer 306. Any number of these chemical tracers can be embedded in the SED Portal System either sharing the same detection layer 306 of the SEDS port/window 300 or dividing the detection layer 306 into zones, much like one Ph strip detects acids, bases, or any combination along the strip's spectrum. Figure illustrates for detection layer is 306 formed as a first zone 311, a second zone 312, third zone 314, and fourth zone 316. The number of zones may be increased or decreased and may be formed from differing material to detect different types of explosives.

Seaport Container Detection
The other looming security vulnerability at seaports could also be a thing of the past with implementation of an explosives or multiplexed with a CBRNE detection portal. Individual boxes/containers 302 in cargo containers shipped to America's ports each and every day could be screened as easily as installing SED System portals and having security quickly inspect them.

Ground Delivery Protection
Though air cargo poses the greatest threat to life from package borne explosives, ground transportation is vulnerable to the use of explosives too, as the Austin bomber attacks revealed. The SEDS System can be implemented with an imbedded detection port/window 300 costing less than a dollar, which will protect carriers, their customers, and the public from package bombs.

The following examples are presented to provide a more complete understanding of The Sirius Explosives Detection System. The specific techniques, combinations, materials, proportions, and reported data vary depending on the chemical detecting substances used and should not be construed as limiting the scope.

In another embodiment, the explosive detection port/cartridge/filter is designed to utilize Exspray. Exspray is a reliable, efficient, direct explosive detection reagent aerosol-based which covers all forms of Group A explosives (e.g. TNT, TNB, etc.), Group B explosives (e.g. Semtex H, RDX, C-4, etc.), and compounds containing the latest terrorist improvised explosives (e.g. peroxide-based). The level of sensitivity (20 nanograms or less) surpasses that of any other comparable product. The testing process is instantaneous; the results appear in seconds. The identification/detection process requires no special training.

In another embodiment, canine can be utilized in a complimentary system to check and recheck all other methods. Dogs offer a practical solution due to their extraordinary ability to detect the faintest odors and trace amounts of explosives and to physically pursue them to their source. The Sirius Solutions Explosive Detection System would “open” the container to the canine and collect vapor or trace particulates in its filter/collection layer for increased concentration and detection by the canines.

An additional embodiment involves the embedding of a chemical called luminol in The Sirius Solutions Explosive Detection System's collection layer/filter for detecting small quantities of explosives and other hazardous substances located on objects. The technique employs a chemical reaction between NO₂ gas and luminol in an aqueous, alkaline solution. Under suitable conditions, the vast majority of common explosive types may be decomposed to produce NO₂ gas that is detected using the apparatus presented in full and incorporated by reference in U.S. patent application 2004/0053421A1. The luminol reaction is known to produce light by a process termed chemiluminescence. This light in turn is detected, to signal the presence of a contraband substance within the container such as any one of many explosives.

An additional embodiment employs an external electronic explosive and hazardous substance detecting device developed by a team from M.I.T., U.S. and sold through a company called, Nomadics. This device is capable of detecting concealed explosives with different sensitivity than trained canines. The device would be used to determine the presence of vapor or trace particulates from an explosive or hazardous substance within a container after The Sirius Solutions Explosion Detection System opens the included container through the System's portal and concentrates the vapors or trace particulates in the System's collection filter material. This separate electronic explosive or hazardous material identifier, called “FIDO,” incorporates a laser and a polymer sensor. Full details can be found at the parent company's website, nomadics.com.

An additional embodiment employs The Frontline Explosives Test Kit (ETK), a drypack application test kit for the detection of explosive residue. The ETK has achieved complete removal of all liquid reagents in detection. The kit would be used to determine the presence of vapor or trace particulates from a contained explosive or hazardous substance after The Sirius Solutions Explosion Detection System opens the included container through the System's portal and concentrates the vapors or trace particulates in the System's collection filter material.

A further embodiment employs a method of determining the presence and underlying molecular identity of trace quantities of explosives or other hazardous materials including chemical and biological warfare agents, with a mass spectrometer. The embodiment would facilitate the analysis by provoking the emanation of trace particulate or vapor identifiers of an explosive or hazardous substance inside a container, piece of luggage, or confined space. The opening of the container, mail or luggage with The Sirius Solutions Explosive Detection Portal would create air flow on which the trace particulates or vapor identifiers would be carried before being collected by the semipermeable filter for increased concentration and spectroanalysis. The mass spectrometer can analyze picogram-sized samples.

What is claimed is:

1. A system for preventing explosions in a protected object which include containers and packages.

2. A system for preventing explosions in a protected object as in claim 1, wherein the protected object is a prepackaged box.

3. A system for preventing explosions in a protected object as in claim 1, wherein the protected object is a uniform loading device.

4. A system for preventing explosions in a protected object which include containers and packages, comprising:
a container being hollow to cover the protected object;
wherein the container includes a window within the surface of the container having a detection layer;
wherein the detection layer includes a nanowire grid to detect explosive material within the container.

5. A system for preventing explosions in a protected object as in claim 4, wherein the protected object is a prepackaged box.

6. A system for preventing explosions in a protected object as in claim 4, wherein the protected object is a cargo container.

7. A system for preventing explosions in a protected object as in claim 4, wherein the protected object is a uniform loading device.

8. A system for preventing explosions in a protected object as in claim 4, wherein the detection layer includes a multitude of zones to detect different explosive devices.
